(19) United States
(12) Patent Application Publication
(10) Pub. No.: US 2008/0031945 A1
(43) Pub. Date: Feb. 7, 2008

(54) GASTRO RETENTIVE DELIVERY SYSTEM

(76) Inventors: Wolfram EISENREICH, Ulm (DE); Thomas FRIEDL, Ochsenhausen (DE); Sebastian HAERTTER, Warthausen (DE)

Correspondence Address: MICHAEL P. MORRIS;BOEHRINGER INGELHEIM CORPORATION, 900 RIDGEBURY ROAD, P. O. BOX 368, RIDGEFIELD, CT, 06877-0368, US

(21) Appl. No.: 11/832,048

(22) Filed: Aug. 1, 2007

(30) Foreign Application Priority Data
Aug. 1, 2006 (EP) 06 118 268

(51) Int. Cl. A61K 9/20

(52) U.S. Cl. 424/465; 424/464

(57) ABSTRACT

The present invention is directed to pharmaceutical composition for the manufacture of a gastro retentive drug delivery system comprising a pharmaceutical formulation and an application condition of the same.

This Application claims the benefit under 35 U.S.C. 119(a) of the filing date of EP 06118268, filed on Aug. 1, 2006, which foreign application is incorporated herein in its entirety by reference.

### BACKGROUND OF THE INVENTION

1. Field of the Invention

The present invention is directed to pharmaceutical compositions for the manufacture of a gastro retentive drug delivery system comprising a pharmaceutical formulation and a defined application condition of the same.

2. Description of the Prior Art

For more than 20 years, development of gastric retained dosage forms was attempted resulting in only a limited number of promising technologies and products on the market.

Gastric retention of more than 6 hours still poses a considerable challenge to the pharmaceutical art.

Subject of the present invention is the development of a gastro retentive drug delivery system providing an extended residence time of the dosage form in the stomach of preferably more than 4 hours. Such a system is useful to improve the bioavailability and the duration of action of drugs.

### SUMMARY OF THE INVENTION

The present invention is directed to pharmaceutical composition for the manufacture of a gastro retentive drug delivery system comprising a pharmaceutical formulation and an application condition of the same.

### BRIEF DESCRIPTION OF THE DRAWINGS

FIG. 1 illustrates a preferred embodiment of the manufacturing process with reference to a flow diagram wherein the manufacture of the extended release tablets of Examples 4 and 5 are exemplarily shown. FIG. 1 shows the detailed process steps and the in process controls performed.

### DESCRIPTION OF THE INVENTION

The invention relates to a gastro retentive drug delivery system enabling a pharmaceutical formulation having an extended residence time in the stomach. Typically, the gastric emptying time is in fasted state in the range from 0-2 hours and in the fed state from 4-6 hours. Purpose of the present invention is to describe a drug delivery system for to provide a pharmaceutical formulation to stay in the stomach for at least 4, preferably 6 hours. Surprisingly it was found that the sustained release formulations of the present invention show stomach residence times of more than 4 hours if taken in fed state. According to the present invention such a combination of a pharmaceutical formulation and a defined application scheme provides a new drug delivery platform technology with an interesting gastro retentive profile for many drugs.

The pharmaceutical formulation which is part of gastro retentive drug delivery system according to the invention has a defined minimum size and combines retarding, swelling and mucoadhesive properties. According to the invention the formulation comprises either one polymer having all three effects or two polymers, one providing the mucoadhesive property, the other one providing a retarding and swelling effect. Preferably the gastro retentive drug delivery system of the present invention comprises a gastro retentive tablet formulation of a defined minimum size, wherein the matrix comprises at least two water swelling polymers and wherein at least one of the at least two polymers is an anionic polymer.

The size of tablet refers to a tablet prior to application by the patient, if not otherwise stated.

The size of the tablet shall be defined according to the 3 dimensions in space, namely its length, its width and its height. It will be acknowledged that the skilled person in the art will not have difficulties in establishing the length, the width, and the height. In case of unusual forms of the tablet, the body of such tablet shall be idealized to the closest mathematical body in order to define length, width, and height. Examples for such idealized mathematical bodies are: cube, cuboid, tetrahedron, hexahedron, octahedron, dodecahedron, icosahedron, prism, ball, ellipsoid, paraboloid, cone, ring, sphere, and the like. These idealized mathematical bodies also may be in a compressed shape.

Typically, the length of a body is the distance between the two subtending points at which the main axis of said body subtends the corresponding subtending surfaces areas of the body. The main axis usually is the medial axis of a shape, an axis around which a geometric rotation body rotates, a symmetrical axis, or an optical axis. For the skilled person in the art it will be an easy to define a main axis of a tablet.

The width of the tablet shall be the longest distance between the two subtending points at which an axis of the body, which is perpendicular to the main axis, subtends the corresponding subtending surfaces areas of the body. The width of the body is equal or smaller than the length and equal or longer than the height.

Finally, the height or thickness is defined as the distance between the two subtending points at which an axis of the body, which is perpendicular to the main axis and perpendicular to the axis that defines the width, subtends the corresponding subtending surfaces areas of the body. The height of the body is equal or smaller than the width.

In less symmetrical bodies, the axis by which the width is defined and the axis by which the height is defined and the axis by which the length is defined need not touch each other, but may be displaced. The same applies for each pair of two axes.

The axes that define the length, the width, and the height typically are perpendicular to each other.

In a cube or in a ball, the axes that define length, the width, and the height are equally long, perpendicular to each other and meet in one point.

In cuboid, the three axes are not all equally long, but again perpendicular to each other and meet in one point.

According to the invention the tablet is characterised in that the length and the width have independent from each other a minimum length which corresponds to at least 7/12, more preferably at least 8/12, more preferably at least 9/12, more preferably at least 10/12, more preferably at least 11/12, more preferably at least 12/12, more preferably at least 13/12, more preferably at least 14/12, more preferably at least 15/12, more preferably at least 16/12, more preferably at least 17/12, more preferably at least 18/12, more preferably at least 19/12, more preferably at least 20/12 of the patient's pyloric diameter.

The human pyloric diameter in average is 12 mm +/−7 mm. All relations of the length or the width to human pyloric diameter shall refer to the average amount of 12 mm in order to calculate absolute amounts of the length and the width.

The tablet for a human patient of normal adult size is characterised in that its length and its width have independent from each other a minimum length of at least 6 mm, preferably at least 7 mm, preferably at least 8 mm, preferably at least 9 mm, preferably at least preferably 10 mm, preferably at least 11 mm, preferably at least 12 mm, preferably at least 13 mm, preferably at least 14 mm, preferably at least 15 mm, preferably at least 16 mm, preferably at least 17 mm, preferably at least 18 mm, preferably at least 19 mm, preferably at least 20 mm. Values are preferred in that the length is longer than the width. The length preferably is at is at least 9 mm, more preferably at least 11 mm. The width is at least 6 mm, preferably at least 7 mm long.

In these embodiments, neither the length nor the width have a maximally preferred length of more than 50 mm, preferably not more 40 mm, preferably not more than 25 mm, preferably not more than 20 mm.

For human children, with smaller pyloric diameter, the length and width of such tablet are correspondingly smaller.

In case the tablet according to the invention is used for an animal the size may differ from that of a human patient according to the ratio of length/width to the animal's pyloric diameter. In case of an animal as patient, the animal preferably is selected from the group of horses, cows, pigs, dogs, cats, rabbits, bunnies, chicken, more preferably it is selected from the group of horses and cows.

In a preferred embodiment of the invention the tablet for a human patient is a round shaped tablet, i.e., a compressed ball having a diameter of at least 9, more preferably at least 11 mm.

In another preferred embodiment, the tablet is an oval shaped tablet having a length of at least 15 mm and a width of at least 7 mm.

Preferred minimum values width×length, both in mm, are:

(7×12); (7×13); (7×14); (7×15); (7×16); (7×17); (7×18); (7×19); (7×20);
(8×12); (8×13); (8×14); (8×15); (8×16); (8×17); (8×18); (8×19); (8×20);
(9×12); (9×13); (9×14); (9×15); (9×16); (9×17); (9×18); (9×19); (9×20);
(10×12); (10×13); (10×14); (10×15); (10×16); (10×17); (10×18); (10×19); (10×20);
(11×12); (11×13); (11×14); (11×15); (11×16); (11×17); (11×18); (11×19); (1×20);
(12×12); (12×13); (12×14); (12×15); (12×16); (12×17); (12×18); (12×19); (12×20);
(13×13); (14×14); (15×15); (16×16); (17×17); (18×18); (19×19); (12×20).

Each pair of which independently is preferred.

The height preferably is at least 1 mm, 2 mm, 3, mm, 4, mm, 5, mm, 6 mm, 7, mm, 8, mm, 9 mm, 10 mm.

To provide a mucoadhesive effect, the invention makes use of “retarding polymers with mucoadhesive properties,” preferably anionic polymers. Without limitation, such polymers may be selected from the group of carboxyalkylcelluloses such as carmellose sodium or carmellose calcium, chondroitin sulfate, acrylic acid polymerisate, pectin, alginates, carrageenans, chitin derivates such as chitosan, preferably acrylic acid polymerisate or chitosan. Among the preferred anionic polymer is an optionally crosslinked acrylic acid polymer. As acrylic acid polymerisate one may use one of the carbomer or CARBOPOL® series, having high molecular weights. Particularly preferred are for example carbomer 941 (CARBOPOL® 71 G, CARBOPOL® 971) and carbomer 934 (CARBOPOL® 974). The content of the optionally crosslinked acrylic acid polymer in the matrix is from about 0.1 wt.-% to about 40 wt.-% and preferably from about 0.1 wt.-% to about 20 wt.-%.

The absolute amount of the retarding polymer preferably is between 0.5 and 600 mg, more preferably 0.5 to 400 mg, more preferably 0.5 to 200 mg or 0.5 to 100 mg. Even more preferred values are between 2 mg and 150 mg, more preferred between 2 mg and 100 mg, more preferably between 2 mg and 50 mg, more preferably between 2 mg and 25 mg.

In one embodiment, the amount is between 2 mg to 600 mg, in another embodiment between 3.9 mg and 400 mg, in another embodiment between 4 and 340 mg, in another embodiment between 4 mg and 340, and still in another embodiment between 5 mg and 300 mg.

To provide a retarding or an increased retarding effect, the formulation according to the invention may comprise a “swelling retarding polymer,” a water swelling substantially neutral polymer. Without limitation, such swelling retarding polymers may be selected from the group of alkylcelluloses, such as, methylcellulose; hydroxyalkylcelluloses, for example, hydroxymethylcellulose (HPMC), hydroxyethylcellulose, hydroxypropylcellulose and hydroxybutylcellulose; hydroxyalkyl alkylcelluloses, such as, hydroxyethyl methylcellulose and hydroxypropyl methylcellulose; other natural, semi-synthetic, or synthetic di-, oligo- and polysaccharides such as galactomannans, tragacanth, agar, guar gum, and polyfructans; ammonio methacrylate copolymers; polyvinylalcohol; polyvinylpyrrolidone, copolymers of polyvinylpyrrolidone with vinyl acetate; combinations of polyvinylalcohol and polyvinylpyrrolidone; polyalkylene oxides such as polyethylene oxide and polypropylene oxide; copolymers of ethylene oxide and propylene oxide; preferably polyethylene oxide and cellulose ether derivatives such as hydroxypropyl methylcellulose and hydroxypropylcellulose, most preferred hydroxypropyl methylcellulose.

Such neutral polymer swells upon contact with aqueous fluid following administration, resulting in a viscous, drug release regulating gellayer. The viscosity of the polymer preferably ranges from 50 to 100,000 mPa.s (apparent viscosity of a 2% aqueous solution at 20° C.).

Preferably, the amount of water swelling polymer in the present formulation ranges from about 10 to about 80% by weight.

The absolute amount of the swelling polymer preferably is between 10 and 1200 mg, preferably between 20 mg and 800 mg, more preferably between 40 mg and 700 mg, more preferably between 50 mg and 400 mg.

In one embodiment the amount is between 20 mg to 1200 mg, in another embodiment between 39 mg and 800 mg, in another embodiment between 40 and 680 mg, in another embodiment between 50 mg and 600 mg.

Preferably, the amount of the swelling polymer is adjusted in that at least the length of the tablet grows in the fed stomach to at least 11/12, more preferably at least 12/12, more preferably at least 13/12, more preferably at least 14/12, more preferably at least 15/12, of the pyloric diameter of the patient, which in average for a human being is at least 12 mm. Preferably the tablet reaches the aforementioned length within less than 3 hours, preferably within less than 2 hours, more preferably within less than 90 minutes and more preferably within less than 60 minutes.

More preferably, the amount of the swelling polymer is adjusted in that in addition to the growing of the length, the width of the tablet grows in the fed stomach to at least 8/12, more preferably at least 9/12, more preferably at least 10/12, more preferably at least 11/12, more preferably at least 12/12, of the diameter of pyloric diameter of the patient, which in average for a human being is at least 12 mm.

Among the substantially neutral polymers hydroxypropylcellulose and hydroxypropyl methylcellulose are preferred.

Different viscosity grades of hydroxypropylcellulose and hydroxypropyl methylcellulose are commercially available. Hydroxypropyl methylcellulose (HPMC) preferably used in the present invention has a viscosity grade ranging from about 50 mPa.s to about 100,000 mPa.s, in particular ranging from about 75 mPa.s to about 20,000 mPa.s and most in particular a viscosity grade of about 100 mPa.s to about 15,000 mPa.s (apparent viscosity of a 2% aqueous solution at 20° C.), e.g., hypromellose 2910, 2208 or 2206 (DOW, Antwerp, Belgium). HPMC type 2208 contains 19-24% by weight methoxy and 4-12% by weight hydroxypropoxy substituents.

Hydroxypropylcellulose having a viscosity higher than 300 mPa.s (apparent viscosity of a 10% aqueous solution at 20° C.) is preferred, in particular hydroxypropylcellulose having a viscosity in the range from about 300 to about 30000 mPa.s, preferably from 4000 to 6500 mPa.s (2% aqueous solutions), e.g., the Klucel series such as Klucel M (Hercules, Wilmington, USA).

According to a preferred embodiment of the present invention the matrix of a gastro retentive tablet formulation comprises or essentially consists of hydroxypropyl methylcellulose, such as hypromellose, and further excipients. The amount of hydroxypropyl methylcellulose is preferably in the range from 10 to 80%, particularly preferred from 15 to 65% most preferred from 20 to 50% by weight. The amount of further excipients is preferably in the range from 80 to 25%, particularly preferred from 75 to 35%, most preferred from 65 to 45% by weight.

Such systems with mucoadhesive, retarding and swelling properties are useful to extend the gastric residence time by adhering them to the gastric mucous membrane. Even though some of the mucoadhesive polymers are effective at producing bioadhesion, it is very difficult to maintain a residence time over several hours with this effect alone because of the rapid turnover of mucus in the gastrointestinal tract.

When using a combination of a neutral and anionic polymer, the ratio of said polymers also may influence the gastro retentive profile of the preparation. Accordingly, such combination facilitates control of the gastro retentive profile of the preparation at will and it will be perspicuous for the skilled person in the art, that the gastro retentive profile may be adjusted via the ratio of said polymers, which is another benefit of the present invention.

According to a preferred embodiment of the present invention a tablet formulation is provided having a matrix that comprises or essentially consists of hydroxypropyl methylcellulose, acrylic acid polymerisate and further excipients. The amount of hydroxypropyl methylcellulose is preferably in the range from 10 to 80%, particularly preferred from 15 to 65%, most preferred from 20 to 50% by weight. The amount of acrylic acid polymerisate is preferably as above-mentioned. The amount of additional excipients is preferably in the range from 80 to 25% particularly preferred from 75 to 35%, most preferred from 65 to 45% by weight.

The tablet formulation of the present invention optionally comprises an active ingredient. Such active ingredient may show pH-dependent solubility, and/or show a limited absorption window in the gastrointestinal tract, and/or be intended for local treatment in the stomach or the small intestine, and/or show low stability in intestinal fluids, and/or degrade by enzymes/bacteria present in the intestine.

However, the tablet formulation also may be a placebo, meaning that the formulation does not comprise an active ingredient. In one embodiment, the tablet excludes pramipexole as active ingredient. In another embodiment the gastro retentive delivery system according to the invention comprises pramipexole.

The formulation according to the invention optionally comprise further excipients, i.e., pharmaceutically acceptable formulating agents, in order to promote the manufacture, compressibility, appearance, and taste of the preparation. These formulating agents comprise, for example, diluents or fillers, glidants, binding agents, granulating agents, anti-caking agents, lubricants, flavors, dyes, and preservatives. Other conventional excipients known in the art can also be included.

The filler may be selected from soluble fillers, for example, sucrose, lactose, in particular lactose monohydrate, trehalose, maltose, mannitol, and sorbitol. Different grades of lactose can be used.

In case of a water soluble active ingredient, more preferably water insoluble fillers, such as starch and starch derivates preferably other than pregelatinized starch, e.g., corn starch, potato starch, rice starch or wheat starch, microcrystalline cellulose, dibasic calcium phosphate dehydrate, and anhydrous dibasic calcium phosphate, preferably corn starch, can be used in addition or instead of the water soluble fillers. The total weight percentage of filler ranges between about 5% and about 75% by weight.

A glidant can be used to improve powder flow properties prior to and during tableting and to reduce caking. Suitable glidants include colloidal silicon dioxide, talc, magnesium trisilicate, powdered cellulose, talc, tribasic calcium phosphate, and the like. Colloidal silicon dioxide is preferably included as a glidant in an amount up to about 2%, preferably about 0.2% to about 0.8%, by weight of the tablet.

A lubricant can be used to enhance release of a tablet from apparatus on which it is formed, for example by preventing adherence to the face of an upper punch (“picking”) or lower punch (“sticking”). Suitable lubricants include magnesium stearate, calcium stearate, canola oil, glyceryl palmitostearate, hydrogenated vegetable oil, magnesium oxide, mineral oil, poloxamer, polyethylene glycol, polyvinyl alcohol, sodium benzoate, sodium lauryl sulfate, sodium stearyl fumarate, stearic acid, zinc stearate, and the like. In one embodiment, magnesium stearate is included as a lubricant in an amount of about 0.1% to about 5%, preferably about 0.5% to about 2%, by weight of the tablet.

Among the optional formulating agents that further may be comprised in the matrix formulation there may be mentioned agents such as polyvidone; copovidone; starch; acacia; gelatin; seaweed derivatives, e.g., alginic acid, sodium and calcium alginate; cellulose, preferably microcrystalline cellulose, cellulose derivatives, e.g., ethylcellulose, hydroxypropylcellulose, hydroxypropyl methylcellulose, having useful dry or wet binding and granulating properties; and antiadherents such as talc and magnesium stearate.

The expression “consisting essentially” is understood in the sense that it does not in principle exclude the presence, in addition to the mandatory components mentioned, of other components, the presence of which does not affect the essential nature of the formulation.

In a preferred embodiment of the present invention the tablet formulation with gastro retentive properties is provided preferably having the following composition: active ingredient 0.01-50% by weight; swelling retarding polymer 10 to 80% by weight, preferably 20-50% by weight; retarding polymers with mucoadhesive properties 0.1-40% by weight, preferably 0.1-20% by weight.

In another preferred embodiment of the present invention the formulation tablet with gastro retentive properties is provided preferably having the following composition: active ingredient 0.05 to 5% by weight; water swelling polymer(s) 10 to 75% by weight; acrylic acid polymerisate 0 to 25% by weight; optional further excipient(s) ad 100% by weight.

Therefore, a particularly preferred tablet formulation according to the invention consists of:
0.1 to 35% by weight of active ingredient thereof,
25 to 65% by weight of hydroxypropyl methylcellulose;
0 to 40% by weight of carboxymethylcellulose sodium;
0 to 75% by weight of corn starch other than pregelatinized starch;
0.1 to 15% by weight of acrylic polymerisate, preferably carbomer 941;
0.5 to 50% by weight of excipients, preferably selected from the group consisting of colloidal silicon dioxide, magnesium stearate, lactose monohydrate, mannitol, microcrystalline cellulose, dibasic anhydrous calcium phosphate,
hydroxyproylcellulose, povidone, copovidone, talc, macrogols, sodium dodecylsulfate, iron oxides and titanium dioxide.

According to the present invention starch, preferably other than pregelatinized starch, preferably corn starch if present, may impart several functions at the same time such as filler, glidant, and the like. However, it may be preferred to exclude starch completely from the tablet formulation according to the present invention, which may be replaced by one or more of the above-mentioned other excipient(s).

It is preferred that no coating is present on the tablet formulation according to the present invention. However, the tablet of the invention may comprise a nonfunctional coating. A nonfunctional coating can comprise a polymer component, for example HPMC, optionally with other ingredients, for example one or more plasticizers, colorants, etc. The term “nonfunctional” in the present context means having no substantial effect on release properties of the tablet, and the coating serves another useful purpose. For example, such a coating can impart a distinctive appearance to the tablet, provide protection against attrition during packaging and transportation, improve ease of swallowing, and/or have other benefits. A nonfunctional coating should be applied in an amount sufficient to provide complete coverage of the tablet. Typically an amount of about 1% to about 10%, more typically an amount of about 2% to about 5%, by weight of the tablet as a whole, is suitable.

The tablets of the present invention can be of any suitable size and shape, for example round, oval, polygonal or pillow-shaped, and optionally bear nonfunctional surface markings. According to the present invention it is preferred that the extended release tablets are white to off-white and of oval or round, biconvex, shape.

In another preferred embodiment of the invention, a tablet is provided having weight in the range of 200 mg to 1500 mg, preferably 390 mg to 1000 mg, more preferably 400 mg to 850 mg and even more preferably of 500 mg to 750 mg.

The gastro retentive delivery system according to the invention comprises the tablets as hereinbefore described together with information about using the same for providing the gastro-retentive effect.

Accordingly, tablets of the invention can be packaged in a container, accompanied by a package insert providing pertinent information such as, for example, dosage and administration information, contraindications, precautions, drug interactions and adverse reactions.

However, the aforementioned approach directed to the composition of the pharmaceutical formulation according to the invention alone may not provide the herein disclosed effect.

It is recommended to apply the tablets of the drug delivery system of the present invention in fed state, meaning after meal. This is as it has been observed that food, particularly fatty acids, prevents emptying of the stomach.

In the context of the present invention the term “fed state” means that patients take the drug at maximum 4 hours, preferably at maximum 3 hours, more preferably at maximum 2 hours, even more preferably at maximum 1 hour, even more preferably at maximum 30 minutes and most preferably just after an ordinary meal (breakfast, lunch, dinner). In an alternative preferred embodiment, the patients may take the tablet while eating. The opposite of fed state is an empty stomach, meaning that the last meal was taken at least 4 hours, more preferably at least 5 hours, more preferably at least 6 hours ago.

Within the context of the present invention “fed” or “fed state” preferably means that a tablet is taken during, just before or just after a meal, more preferably during or just after a meal.

Furthermore, the present invention is preferably directed to a method of manufacturing the extended release tablet formulations via a direct compression process comprising the steps of

(1) producing an active ingredient trituration by preblending it with a portion of water swelling polymer(s) and/or further excipient(s) in a mixer;
(2) premixing the active ingredient trituration of step (1), the main portion of the water swelling polymer(s) and/or excipients in a mixer to obtain a pre-mixture;
(3) optionally dry screening the pre-mixture through a screen in order to segregate cohesive particles and to improve content uniformity;
(4) mixing the pre-mixture of step (2) or (3) in a mixer, optionally by adding remaining excipients to the mixture and continuing mixing; and
(5) tableting the final mixture by compressing it on a suitable tablet press to produce matrix tablets.

Also other processes can be applied to the manufacturing of tablets according to the invention, like conventional wet granulation and roller compaction. In case of wet granulation the active ingredient may be granulated with suitable fillers, like e.g., starches other than pregelatinized starch, microcrystalline cellulose, lactose monohydrate or anhydrous dibasic calcium phosphate, and wet binding agents, like e.g., hydroxypropyl methylcellulose, hydroxypropylcellulose, povidone, copovidone, and starch paste, leading to a active ingredient concentrate, which after drying and dry screening is mixed with the main fraction of gel forming excipients, like all the above described retarding principles.

In case of roller compaction, or in other words dry granulation, either a premix of active ingredient with part of the excipients used in the direct compression process, or the complete mixture containing all excipients, is processed through a conventional roller compactor to form ribbons, which are thereafter screened down to granules which are finally mixed with other excipients, like glidants, lubricants and antiadherents.

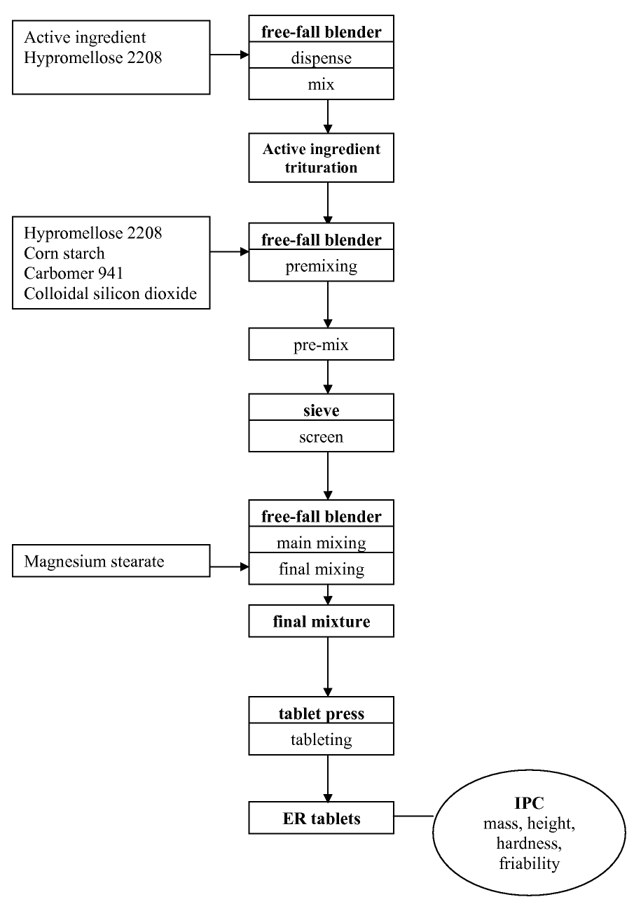

FIG. 1 illustrates a preferred embodiment of the manufacturing process with reference to a flow diagram wherein the manufacture of the extended release tablets of Examples 4 and 5 are exemplarily shown. FIG. 1 shows the detailed process steps and the in process controls performed.

Process step (1) is directed to the active ingredient trituration, where the active ingredient is preblended with a portion of the polymer, in this case hydroxypropyl methylcellulose, in a commonly known mixer. In the flow chart a TURBULA® free fall mixer or blender is used. The mixing time is several minutes, in the present case preferably 10 min.

In process step (2) according to the flow chart a premixing is performed, wherein the active ingredient trituration and the main portion of the water swelling polymer(s) and excipients are premixed for several minutes to obtain a pre-mix. In the present case the main portion of hydroxypropyl methylcellulose (hypromellose), corn starch, carbomer 941 and colloidal silicon dioxide are premixed for 5 min. in the above-mentioned TURBULA® mixer or blender.

According to the following process step (3) a dry screening may optionally take place. The pre-mixture may be manually screened through a screen, for example a 0.8 mm mesh size screen, in order to segregate cohesive particles and to improve content uniformity.

In the subsequent process step (4) the main mixing step is performed according to which the components are mixed for several minutes, preferably 5 min. in the TURBULA® mixer after screening. Optionally further excipients may be added at this time, in the flow chart the component magnesium stearate is added to the main mixture, and further mixing for several minutes, e.g., 3 min., in the TURBULA® mixer is performed (final mixing) to obtain the final mixture.

Process step (5) of the process according to the present invention is the tableting. The final mixture is compressed on a suitable tablet press to produce, for example, oval shaped matrix tablets (ER tablets=extended release tablets). In order to control and maintain the required quality, the obtained matrix tablets are subjected to the following in-process controls: tablet mass, hardness, tablet height, and friability.

The obtained tablets of the present invention may then be filled, for example, into High Density Polyethylene (HDPE) bottles. The bottles are closed tightly with screw caps and appropriately labelled, whereby all packaging and labelling activities are performed according to cGMP regulations. Alternatively, a blister type packaging can be used, e.g., using aluminium/aluminium foil blisters.

Furthermore, the tablets of the present invention may be manufactured via a direct compression, wet or dry granulation process.

### FORMULATION EXAMPLES

Placebo tablets were prepared with the following composition

| | | |
|---|---|---|
| Hypromellose 2208 | 112.50 | mg |
| Maize starch | 114.75 | mg |
| Carbomer 941 | 15.00 | mg |
| Iron oxide black | 5.00 | mg |
| Colloidal anhydrous silica | 1.50 | mg |
| Magnesium stearate | 1.25 | mg |
| Total weight placebo tablet | 250.00 | mg |
| Hypromellose 2208 | 225.0 | mg |
| Maize starch | 249.5 | mg |
| Carbomer 941 | 15.0 | mg |
| Iron oxide black | 5.0 | mg |
| Colloidal anhydrous silica | 3.0 | mg |
| Magnesium stearate | 2.5 | mg |
| Total weight placebo tablet | 500.0 | mg |
| Hypromellose 2208 | 315.0 | mg |
| Maize starch | 321.3 | mg |
| Carbomer 941 | 42.0 | mg |
| Iron oxide black | 14.0 | mg |
| Colloidal anhydrous silica | 4.2 | mg |
| Magnesium stearate | 3.5 | mg |
| Total weight placebo tablet | 700.0 | mg |

Further examples with active ingredients are

| | | |
|---|---|---|
| Active ingredient | 0.750 | mg |
| Hypromellose 2208 (Methocel K 15 M) | 157.500 | mg |
| Corn starch | 183.700 | mg |
| Carbomer 941 (Carbopol ® 71 G) | 3.500 | mg |
| Colloidal Silicon dioxide | 2.800 | mg |
| Magnesium stearate | 1.750 | mg |
| Total weight matrix tablet | 350.000 | mg |
| Active ingredient | 4.500 | mg |
| Hypromellose 2208 (Methocel K 15 M) | 225.000 | mg |
| Corn starch | 250.000 | mg |
| Carbomer 941 (Carbopol ® 71 G) | 15.000 | mg |
| Colloidal Silicon dioxide | 3.000 | mg |
| Magnesium stearate | 2.500 | mg |
| Total weight matrix tablet | 500.000 | mg |
| Active ingredient | 0.750 | mg |
| Hypromellose 2208 (Methocel K 15 M) | 157.500 | mg |
| Corn starch | 174.600 | mg |
| Carbomer 941 (Carbopol ® 71 G) | 14.000 | mg |
| Colloidal Silicon dioxide | 1.400 | mg |
| Magnesium stearate | 1.750 | mg |
| Total weight matrix tablet | 350.000 | mg |
| Active ingredient | 1.500 | mg |
| Hypromellose 2208 | 315.000 | mg |
| Corn starch | 349.200 | mg |
| Carbomer 941 | 28.000 | mg |
| Colloidal Silicon dioxide | 2.800 | mg |
| Magnesium stearate | 3.500 | mg |
| Total weight matrix tablet | 700.000 | mg |
| Active ingredient | 0.750 | mg |
| Hypromellose 2208 (Methocel K 15 M) | 180.000 | mg |
| Carboxymethylcellulose sodium | 100.000 | mg |
| Lactose monohydrate (200 mesh) | 50.000 | mg |
| Microcrystalline cellulose (grade PH 101) | 65.750 | mg |
| Colloidal silicon dioxide | 1.500 | mg |
| Magnesium stearate | 2.000 | mg |
| Total weight matrix tablet | 400.000 | mg |
| Active ingredient | 100.000 | mg |
| Hydroxypropylcellulose | 270.000 | mg |
| Carboxymethylcellulose sodium | 60.000 | mg |
| Lactose monohydrate (200 mesh) | 50.000 | mg |
| Microcrystalline cellulose (grade PH 101) | 99.000 | mg |
| Carbomer 941 (Carbopol ® 71 G) | 6.000 | mg |
| Colloidal silicon dioxide | 3.000 | mg |
| Magnesium stearate | 12.000 | mg |
| Total weight matrix tablet | 600.000 | mg |
| Active ingredient | 0.750 | mg |
| Hypromellose 2208 (Methocel K 15 M) | 175.000 | mg |
| Carboxymethylcellulose sodium | 87.500 | mg |
| Lactose monohydrate (200 mesh) | 45.500 | mg |
| Microcrystalline cellulose (grade PH 101) | 24.100 | mg |
| Carbomer 941 (Carbopol ® 71 G) | 14.000 | mg |
| Colloidal silicon dioxide | 1.400 | mg |
| Magnesium stearate | 1.750 | mg |
| Total weight matrix tablet | 350.000 | mg |
| Active ingredient | 200.00 | mg |
| Hypromellose 2208 (Methocel E50 LV) | 300.00 | mg |
| Lactose monohydrate | 190.00 | mg |
| Carbomer 941 (Carbopol ® 71 G) | 37.500 | mg |
| Colloidal silicon dioxide | 7.50 | mg |
| Magnesium stearate | 15.00 | mg |
| Total weight matrix tablet | 750.000 | mg |
| Active ingredient | 100.00 | mg |
| Hypromellose 2910 (Methocel E50 LV) | 100.00 | mg |
| Microcrystalline cellulose | 215.00 | mg |
| Sodium alginate | 25.00 | mg |
| Organic acid | 50.00 | mg |
| Colloidal silicon dioxide | 2.50 | mg |
| Magnesium stearate | 7.50 | mg |
| Total weight matrix tablet | 500.00 | mg |
| Active ingredient | 100.00 | mg |
| Hypromellose 2208 (Methocel K 100 LV) | 150.00 | mg |
| Microcrystalline cellulose | 235.00 | mg |
| Carbomer 941 (Carbopol ® 71 G) | 5.00 | mg |
| Colloidal silicon dioxide | 2.50 | mg |
| Magnesium stearate | 7.50 | mg |
| Total weight matrix tablet | 500.000 | mg |

### Gastro Retentive Effect

The gastro retentive effect was proven with magnetically marked tablets of the aforementioned type (Placebo tablet of 250 mg and 500 mg weight with incorporation of Fe3O4-Magnetit.)

The tablets were applied to patients and the GI-transit was monitored via the magnetic properties of the tablets. The decline of aligned magnetic moment was correlated with in vivo disintegration.

In a randomized, open, four-way changeover magnetic marker monitoring study the gastro-intestinal transit and in vivo disintegration process of two differently sized extended release matrix tablets containing Fe3O4 (E172) was evaluated at 8 healthy volunteers (4 males 4 females). The volunteers were given the magnetically marked tablets in fasted and fed state. It was observed during routine in-vitro dissolution tests that the gastro retentive dosage form starts to swell after contact with fluids and that it starts to float on the top of the dissolution media. The swollen tablets have obviously a lower density than water.

The results showed that in particular tablets show a gastro retentive effect of more than 4 hours if taken in fed state. So it could be shown that the mean residence time in fed state was nine times longer than given without a meal.

Additionally it could be shown that while round tablets of 9 mm diameter (=length and width are each 9 mm) and 4.7 mm height with a weight of 250 mg (=small tablet) already had a residence time in the stomach of more than 5 hours if taken after meal, larger tablets of oval shape and 16.2 mm length, 7.9 mm width and 5.3 mm height, with a weight of 500 mg (=large tablet) even showed a residence time of more than 8 hours when taken under the same conditions.

The average results measured for 8 adult patients are outlined in the following table:

TABLE 1
Results for gastric residence time of the small tablet
given with or without food:

| | Mean residence time |
|---|---|
| Fasted state | 37 minutes |
| Fed state | 325 minutes |

TABLE 2
Results for the gastric residence time of the small
and large tablet given after a meal:

| | Mean residence time |
|---|---|
| Small tablet | 325 minutes |
| Large tablet | 570 minutes |

Accordingly, the results show that the large tablet stays significantly longer in the stomach.

1. A tablet comprising an active ingredient, wherein the length of the tablet prior to swallowing by a patient is 7/12 to 20/12 of the patient's pyloric diameter, and wherein the length of the tablet after swallowing in fed state grows in the stomach to 11/12 to 15/12 of the patient's pyloric diameter.

2. A tablet according to claim 1, wherein the width of the tablet is not larger than the length of the tablet, and wherein the width of the tablet prior to swallowing is 7/12 to 20/12 of the patient's pyloric diameter, and wherein the width of the tablet after swallowing in fed state grows in the stomach to 8/12 to 12/12 of the patient's pyloric diameter.

3. A tablet according to claim 1, wherein the tablet comprises 0.01-50% by weight of an active ingredient, 10 to 80% by weight of a swelling retarding polymer, and 0.1-40% by weight of retarding polymers with mucoadhesive properties.

4. A tablet according to any of claims 3, wherein the swelling retarding polymer is an anionic polymer.

5. A tablet according to claim 3, wherein the swelling retarding polymer is selected from the group consisting of carboxyalkylcelluloses, chondroitin sulfate, acrylic acid polymerisate, pectin, alginates, carrageenans, and chitin derivates.

6. A tablet according to claim 3, wherein the swelling retarding polymer is a water swelling substantially neutral polymer.

7. A tablet according to claim 3, wherein the swelling retarding polymer is selected from the group consisting of alkylcelluloses, hydroxyalkylcelluloses; hydroxyalkyl alkylcelluloses, natural, semi-synthetic, or synthetic di-, oligo- and polysaccharides; ammonio methacrylate copolymers; polyvinylalcohol; polyvinylpyrrolidone, copolymers of polyvinylpyrrolidone with vinyl acetate; combinations of polyvinylalcohol and polyvinylpyrrolidone; polyalkylene oxides; copolymers of ethylene oxide and propylene oxide; and cellulose ether derivatives.

8. A tablet according to claim 3, further comprising excipients selected from the group of consisting of diluents, fillers, glidants, binding agents, granulating agents, anti-caking agents, lubricants, flavors and dyes.

9. A tablet comprising an active ingredient, wherein the length of the tablet prior to swallowing by the patient is 7 mm to 20 mm, and wherein the tablet comprises 0.01-50% by weight of an active ingredient, 10-80% by weight of a swelling retarding polymer, and 0.1-40% by weight of retarding polymers with mucoadhesive properties.

10. A tablet according to claim 9, wherein the width of the tablet is not larger than the length, and wherein the width of the tablet prior to swallowing is 7 mm to 20 mm.

11. A tablet according to claim 9, wherein the swelling retarding polymer is an anionic polymer.

12. A tablet according to claim 9, wherein the swelling retarding polymer is selected from the group consisting of carboxyalkylcelluloses, chondroitin sulfate, acrylic acid polymerisate, pectin, alginates, carrageenans, and chitin derivates.

13. A tablet according to claim 9, wherein the swelling retarding polymer is a water swelling substantially neutral polymer.

14. A tablet according to claim 9, wherein the swelling retarding polymer is selected from the group consisting of alkylcelluloses, hydroxyalkylcelluloses; hydroxyalkyl alkylcelluloses, natural, semi-synthetic, or synthetic di-, oligo- and polysaccharides; ammonio methacrylate copolymers; polyvinylalcohol; polyvinylpyrrolidone, copolymers of polyvinylpyrrolidone with vinyl acetate; combinations of polyvinylalcohol and polyvinylpyrrolidone; polyalkylene oxides; copolymers of ethylene oxide and propylene oxide; and cellulose ether derivatives.

15. A tablet according to claim 9, further comprising excipients selected from the group of consisting of diluents, fillers, glidants, binding agents, granulating agents, anti-caking agents, lubricants, flavors and dyes.

16. A method of providing a residence time of a tablet in a patient's fed stomach of preferably more than 4 hours, wherein the patient is given a tablet according to claim 1.

17. Method according to claim 16, wherein the patient is a human being.

18. Method according to claim 16, wherein the patient is a human adult.

19. Method according to claim 16, wherein the patient is a human child.

20. Method according to claim 16, wherein the patient is an animal selected from the group consisting of horses, cows, pigs, dogs, cats, rabbits, bunnies, and chickens.
